(19) United States
(12) Patent Application Publication
(10) Pub. No.: US 2010/0303877 A1
(43) Pub. Date: Dec. 2, 2010

(54) Controlled Release Hydrogel Films

(75) Inventors: Richard B. Timmons, Arlington, TX (US); Wen Chyan, Denton, TX (US); Dhiman Bhattacharyya, Arlington, MA (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/791,845

(22) Filed: Jun. 1, 2010

Related U.S. Application Data
(60) Provisional application No. 61/183,016, filed on Jun. 1, 2009.

(51) Int. Cl. A01N 25/00; C08L 79/02; A61K 9/00; C08K 3/08; A61K 33/38; A01N 59/16; A01P 15/00; A01P 1/00; A61P 31/00

(52) U.S. Cl. 424/405; 524/612; 424/484; 524/440; 424/618; 977/773; 977/915

(57) ABSTRACT

The present invention provides hydrogels and methods of making hydrogels with precisely controlled levels of chemical compositions by mixing one or more monomers in a plasma reactor; polymerizing the one or more monomers into a polymer; crosslinking the polymer to form a hydrogel; immersing the hydrogel in a first solution; and adsorbing one or more solute species from the solution, wherein the one or more solute species are released at controlled rates.

### CROSS-REFERENCE TO RELATED APPLICATIONS

This application claims priority to U.S. Provisional Application Ser. No. 61/183,016, filed Jun. 1, 2009, the contents of which is incorporated by reference herein in its entirety.

### TECHNICAL FIELD OF THE INVENTION

The present invention relates in general to the fabrication of materials with controlled release hydrogel films, specifically to compositions of matter and methods of making and fabrication of materials with hydrogel films synthesized by a gas phase plasma enhanced chemical vapor deposition processes (PECVD).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with compositions of matter and methods of making and fabrication of materials with hydrogel films synthesized by a gas phase PECVD.

Technology to improve controlled delivery of drugs and other materials such as antimicrobial agents represents an increasingly important need at the present time. This need is driven, in large part, by the continued development of new surgical procedures and new drugs, coupled with the demographics of a progressively aging population and their accompanying medical needs. In recognition of these facts, a variety of technologies are being evaluated with respect to their utility in providing much needed improvements to controlled delivery of drugs and other agents. Certainly, one of the more actively studied technologies considered for this purpose involves the use of hydrogels, particularly those materials which are responsive to stimuli, such as temperature and pH changes. The present invention describes a number of entirely new capabilities involving hydrogels to achieve improved controlled delivery of materials.

Controlled release of silver ions has been selected to illustrate the utility of the present invention. The selection of silver is based on the widely documented efficacy of silver as a potent antimicrobial agent in a large variety of applications. In particular, reduction of implant-related infections represents a major medical research goal at the present time. The fact that the number of implant associated infections now approximate 1 million per year, with directly associated medical treatment costs exceeding $3 billion dollars per year, provides a quantitative measure of the magnitude of this problem. Furthermore, the rapidly increasing use of implants, coupled with the development of bacterial resistance to systemically administered antibiotics, provide a fairly clear indication that, in the absence of technologically improved prevention methods, the prevalence of implant associated infections can be expected to increase even more rapidly in the immediate future.

In light of above considerations, it is not surprising to note continued research emphasis to combat device related infections. Much of this effort has focused on attempts to disrupt or prevent biofilm formation on implant surfaces. The biofilms provide a safe harbor to bacteria from antibiotics such that bacteria within the biofilms are often unaffected or incompletely destroyed by antibiotics. Biofilms also increase the metabolic rate of bacterial cell processes and increase their growth rate. In large part, recent studies have focused on coatings that can release antimicrobial agents, such as those involving antibiotics, antibodies and nitric oxide, in efforts to eliminate or reduce biofilm formation. One of the most widely explored agents to control antimicrobial activity is that involving the release of silver ions.

Silver ions are known to exhibit strong antimicrobial activity against an unusually broad range of bacteria and fungi, at concentrations which are nontoxic to mammalian tissue. For example, cytotoxicity of mammalian cells to Ag⁺ is believed to occur only above silver ion concentrations of 10 mg per liter, while effective antimicrobial activity is observed at Ag⁺ levels below as micrograms per liter. In general, this antibacterial activity is believed to arise from the thermodynamically favorable co-ordination of silver ions with the numerous nucleophilic functionalities (eg. nitrogen, oxygen and sulfur containing groups) readily available in bacterial proteins and on cell membranes. It is believed that the combined disruption of the cell membranes by the silver ions, coupled with subsequent silver ion-protein (particularly Ag⁺-enzyme) interactions, leads to cell death. In addition, silver ions can displace other metal ions required for cell survival. It is also believed that Ag⁺ ions interact with bacterial DNA thus preventing cell reproduction. Given the broad scope of these interactions, it is not surprising to note that the bactericidal and bacteriostatic activity of silver ions have been reported to be effective against a wide range of bacteria, including organisms most directly associated with implant infections. Furthermore, given the nature of the Ag⁺ bactericidal activity, it is generally believed that development of bacterial resistance to silver ion exposure is unlikely. In light of the growing widespread concern of bacterial resistance to antibiotics, this is a particularly significant consideration at this point in time.

Extensive literature, patents, and patent applications describing studies of silver associated antimicrobial activity. A wide variety of approaches have been employed for this purpose. In such studies, the silver is generally released from conventionally synthesized organic polymeric matrices. A wide variety of polymeric materials, synthesized using conventional solution polymerization methods, have been examined, including hydrogels. In contrast, the present invention involves a gas phase synthesis of polymeric hydrogels in which a number of significant new advantages with respect to controlled release considerations have been discovered.

### SUMMARY OF THE INVENTION

The present invention provides a process to synthesize hydrogels with precisely controlled levels of chemical compositions by mixing one or more monomers in a plasma reactor; polymerizing the one or more monomers into a polymer; crosslinking the polymer to form a hydrogel; immersing the hydrogel in a first solution; adsorbing one or more solute species from the solution, wherein the one or more solute species are released at controlled rates; immersing the hydrogel in a second solution; adsorbing one or more capping agents from the second solution, wherein the one or more capping agents modify the release rate of the one or more solute species.

The present invention provides a hydrogel with precisely controlled levels of active agent. The hydrogel includes a mixture of one or more monomers polymerized in to a polymer in a plasma reactor with one or more crosslinks connecting the polymer to form a hydrogel; one or more solute species in connected to the polymer, wherein the one or more solute species are released at controlled rates from the polymer; one or more capping agents in connected to the one or more solute species, the polymer, or both to modify the release rate of the one or more solute species.

The present invention involves the use of hydrogel films synthesized by a gas phase plasma enhanced chemical vapor deposition processes (PECVD). The drug, or agent, whose controlled release is desired, is then absorbed into the plasma-synthesized hydrogel film as achieved, for example, via a simple solution immersion process. Finally, in one important embodiment of this invention, a second plasma polymerization deposition in carried out in which a capping layer is deposited on the hydrogel/drug (or other agent)-containing composite.

The present invention uses gas phase plasma enhanced PECVD process to generate the hydrogel films offers significant advantages in terms of providing completely conformal coatings, in which both control of the chemical composition and precise control of the film thickness of the hydrogel are inherently available. In particular, the use of a variable duty cycle pulsed plasma, in lieu of the conventional continuous-wave operational mode, permits achieving a unique level of combined compositional controllability and film thickness.

The level of cross-linking present in the hydrogels is precisely controllable during the PECVD synthetic step via simple variation of the ratio of the plasma on to plasma off times employed during the deposition.

The extent of drug (or other agent) loading of the hydrogel films can be precisely varied through different combinations of the cross-link density and thickness of the hydrogel films.

The use of capping layers of the present invention provides a very important added level of control to the release dynamics of these composite films. These capping layers, in this case deposited by a second PECVD step, are also precisely controlled with respect to both cross-link density and film thickness. Furthermore, the release rates observed are strongly dependent on the surface energies of the capping layers with respect to their level of wettability. These surface energy variations are readily controlled by appropriate choice of monomer, or monomer mixtures, for the plasma polymerization step involving deposition of the capping layer.

The present invention provides silver nanoparticles formed inside the hydrogels by spontaneous reduction of the silver ions to elemental form, following the silver ion absorption into the films. The presence of these silver nanoparticles plays a major role in helping provide the long term, controlled release of silver achieved through this invention. A related discovery showed that the silver nanoparticles within the hydrogels can also be spontaneously reduced to silver ions when in contact with the hydrogel matrix and exposed to oxygen.

The present invention provides plasma synthesized hydrogel films are stimuli sensitive, as shown with respect to changes in drug (or agent) release rates with changes in temperature. Furthermore, it is possible to vary, in highly controlled fashion, the thermoresponsive hydrogel temperatures by use of mixed monomers during the initial PECVD deposition process.

### BRIEF DESCRIPTION OF THE DRAWINGS

For a more complete understanding of the features and advantages of the present invention, reference is now made to the detailed description of the invention along with the accompanying figures and in which:

FIG. 1 is an image of the PECVD processing was carried out in a bell jar type reactor.

FIG. 2 is a schematic drawing of the analytical system employed.

FIG. 3 is a potentiometric derived calibration curve, from solutions of known silver concentrations, obtained by sequential dilutions of a AgNO₃ standard solution.

FIG. 4 illustrates the high sensitivity of atomic absorption, the GFAA measurements were made with solutions that were dilutions of the potentiometry solutions, with concentrations ranging from 1 to 5 ppb.

FIG. 5 is an image comparing GFAA results, using these diluted solutions, with the predicted Ag⁺ concentrations, calculated from the dilution process for the potentiometric samples provided confirmation of the quantitative agreement between the GFAA and emf data.

FIG. 6A shows the well silver nanoparticles, as being extremely well dispersed and of relatively uniform size. FIG. 6B is a higher resolution TEM pictures of one of the nanoparticles. FIG. 6C is an image of X-Ray fluorescence emissions from these particles.

FIG. 7 shows the variation of total Ag⁺ loading obtained for these 500 nm thick films of compositions A, B, and C.

FIG. 8 is an image of films of compositions A, B, and C, produced under the same three plasma conditions mentioned in example 1, having thicknesses of 220 and 500 nm.

FIG. 9 is an image showing the kinetic of the silver release for measurements made with a composition A sample of 500 nm thickness.

FIG. 10 is an image of the release rates from 500 nm thick sample C films.

FIG. 11 is an image of a graph showing Ag release as a function of time.

FIG. 12 illustrates the correlation of release rates and film cross-link densities, the decrease in silver release from the capping layers becomes increasingly more pronounced in the sequence A<B<C, i.e. as the cross-link density increases.

FIG. 13 is an image that demonstrates that both the initial surge release and subsequent slow zero order release of silver ions was decreased, relative to the uncapped 500 nm control.

FIG. 14 is an image that demonstrates the silver release rates observed for these various films at the two temperatures employed, as observed over a 7 day period.

FIG. 15 is a graph of the variation in silver release rates with increased temperature.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a process to synthesize hydrogels with precisely controlled levels of chemical compositions by mixing one or more monomers in a plasma reactor; polymerizing the one or more monomers into a polymer; crosslinking the polymer to form a hydrogel; immersing the hydrogel in a first solution; adsorbing one or more solute species from the solution, wherein the one or more solute species are released at controlled rates; immersing the hydrogel in a second solution; adsorbing one or more capping agents from the second solution, wherein the one or more capping agents modify the release rate of the one or more solute species.

The process may further include the step of adjusting a variable duty cycle pulsed plasma, a continuous wave plasma, or both to polymerize the one or more monomers into the polymer to control a polymer cross-link density and a thickness.

The one or more solute species comprise one or more bioactive agents, active agents, drugs or antimicrobial agents. The one or more solute species comprise one or more silver ions that are spontaneously reduced to one or more elemental silver particles after adsorption by the hydrogel and are nanosized particles, having diameters of less than 50 nm, more preferably diameters of 1-75 nm. However the size may be 1, 2, 3, 4, 5, 6, 7, 8, 9, 10, 15, 20, 25, 30, 35, 40, 45, 50, 55, 60, 65, 70, 75 and incremental variations thereof. The one or more monomers, a plasma deposition variable or both can be varied to control a behavior at a lower critical solution temperature over the temperature range of approximately 30° C. to 60° C. The one or more capping agents has a surface energy ranging from highly non-wettable (hydrophobic) to highly wettable (hydrophilic) and modify the release rate of the one or more solute species to eliminates any initial surge release of the one or more solute species.

The present invention provides a controlled drug (or other agent) release, as obtained using thin hydrogel films synthesized by gas phase plasma polymerization. The general utility of this new approach is illustrated here with the controlled release of Ag⁺ ions, although it is clear from the descriptions provided that this same general new technology would be easily applicable to controlled release of a wide range of other compounds or materials. Although conventionally synthesized hydrogels containing absorbed silver ions have been previously examined, this is the first time gas phase, plasma generated hydrogel films have been employed for this purpose. The use of plasma synthesized polymeric hydrogel films offer a number of interesting practical, and functional, advantages not readily available via conventional solution based synthetic techniques. Among these considerations, we note that the plasma approach involves a single step, gas phase deposition process, and thus provides an ideal way to generate conformal films having precisely controlled film thickness. Additionally, the use of variable duty cycle pulsed plasmas to generate the hydrogel films, allows unusually simple and direct control over the extent of cross-link density in the synthesized hydrogel films. This, in turn, provides a convenient method to wide-ranging control of Ag⁺ release rates. Finally, in a further embodiment of this invention the use of a gas phase plasma deposited capping layer on the initial hydrogel/silver containing composite provides an extraordinary beneficial level of controlled release.

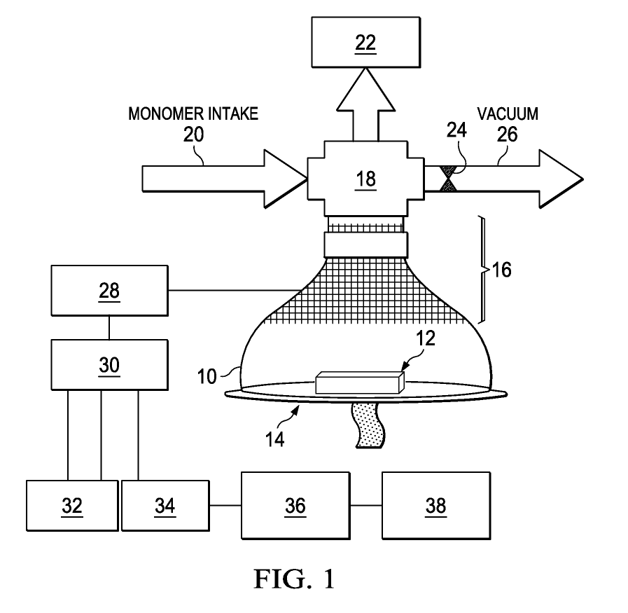

FIG. 1 is an image of the PECVD processing was carried out in a bell jar type reactor. The PECVD processing was carried out in a bell jar type reactor, FIG. 1. FIG. 1 shows a bell jar reactor 10 with a substrate 12 positioned on a ground electrode 14 and below the hot electrode region 16. The hot electrode region 16 is in communication with the upper region 18 that is connected to a thermal intake 20, a pressure transducer 22 and a butterfly valve 24 connecting to a vacuum 26. The hot electrode region 16 is connected to a matching unit 28, a bidirectional coupler 30 connected to an oscilloscope 32 and an amplifier 34. The amplifier 34 is in turn connected to a function generator 36 and a pulse generator 38. This chamber was employed to polymerize low molecular weight monomers which are deposited as thin hydrogel films during operation of the plasma discharges. The discharges were operated under both continuous wave and variable duty cycle pulsed plasma operation, using 13.56 MHz radio frequency power input. Although RF power input was employed in this work, those skilled in the art realize that a wide variety of additional power inputs could also be employed for this purpose, such as microwave, AC and DC electrical discharges, photochemical and radiation methods, etc (e.g., other radio frequency power input may be used 5-20 MHz). The films were deposited on a wide range of substrates which included glass, silicon, polymers, and fabrics. In fact, given the gas phase synthetic process involved, these films can be uniformly and conformally deposited on any solid substrate.

The general procedure employed involved the synthesis initial polymeric hydrogel films having controlled film compositions and a range of film thickness. These films were deposited on a solid substrate, typically a sterile plastic coverslip. The composition controllability of the film was achieved by appropriate control of the plasma variables during the plasma polymerization step. The film thickness, for a given composition, was varied simply by changing the plasma deposition times employed. Subsequently, the films were immersed in a concentrated silver nitrate solution to permit absorption of the silver ions into the hydrogel. After soaking, the films were removed from the silver nitrate solution and washed briefly with deionized water to remove any adsorbed ions. At this point some films were employed to study the rates of silver release from the hydrogel matrices. Additionally, some of the silver nitrate containing films were subjected to a second PECVD step in which a polymer layer was deposited on top of the hydrogel-silver composite film. In some cases, this second layer, hereafter identified as a capping layer, was another hydrogel film but, in other cases, it was a non-hydrogel, plasma generated polymeric film. As in the case of the original hydrogel film, it was possible to control both the composition and thickness of the capping layer by simple control of plasma variables and deposition times.

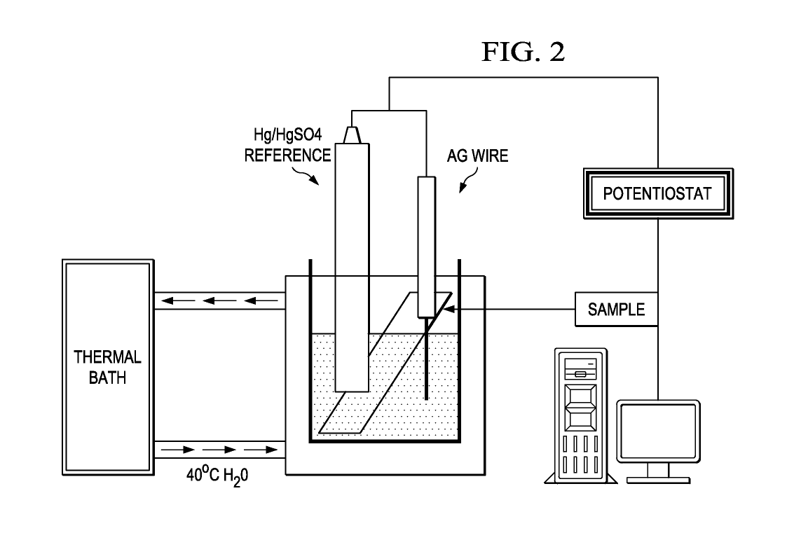

An electrochemical process was developed to monitor the release rates of silver ions from the hydrogel-silver containing composite films. FIG. 2 provides a schematic drawing of the analytical system employed. Basically the process involves a measurement of the cell potential developed between the silver ions released from the film and the silver electrode, as measured against a reference electrode. The Ag⁺ potentiometry is based on the reduction/oxidation equilibrium of Ag⁺ ions with a Ag(s) sensing electrode. The Ag⁺/Ag redox potential was measured with respect to a Hg/Hg₂SO₄ reference electrode. Given that the standard reduction potentials for these half-reactions are 0.799 and 0.614 V, respectively, versus the standard hydrogen electrode, the Nernst equation applicable to these measurements can be summarized as:

$$\begin{matrix}{E}_{cell} = [ 0.799 V - \frac{0.05916}{1} log ( \frac{1}{[ Ag + ]} ) ] - \\ [ 0.614 V - \frac{0.05916}{2} log ( [ S {O}_{4}^{2 -} ] ) ]\end{matrix}$$

$${E}_{cell} = 0.05916 \times log ( [ {Ag}^{+} ] ) + 0.159$$

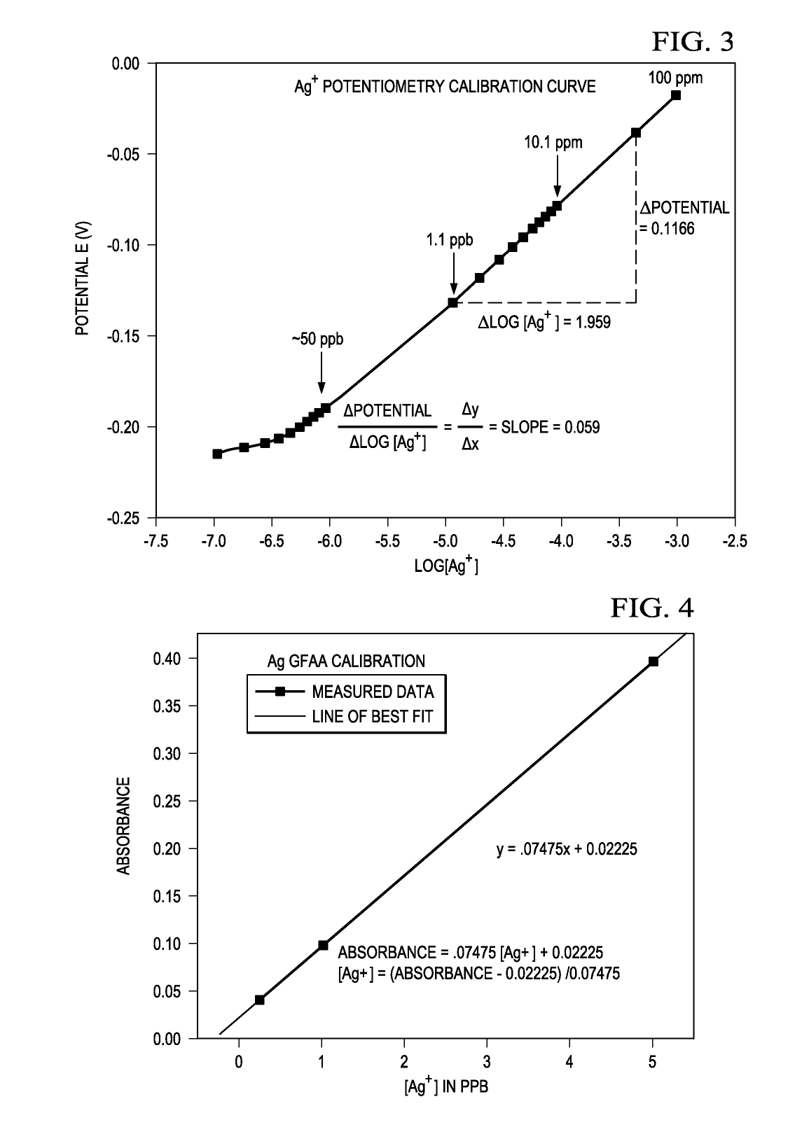

FIG. 3 is a potentiometric derived calibration curve, from solutions of known silver concentrations, obtained by sequential dilutions of a AgNO₃ standard solution. As shown in this figure, the measured cell emfs vary linearly with log [Ag+] over concentrations ranging from 50 ppb to 100 ppm, with the straight line having the requisite slope of 0.059. The unusually wide linear dynamic range provided by this approach proved especially useful in monitoring the Ag⁺ release rates from the hydrogel films.

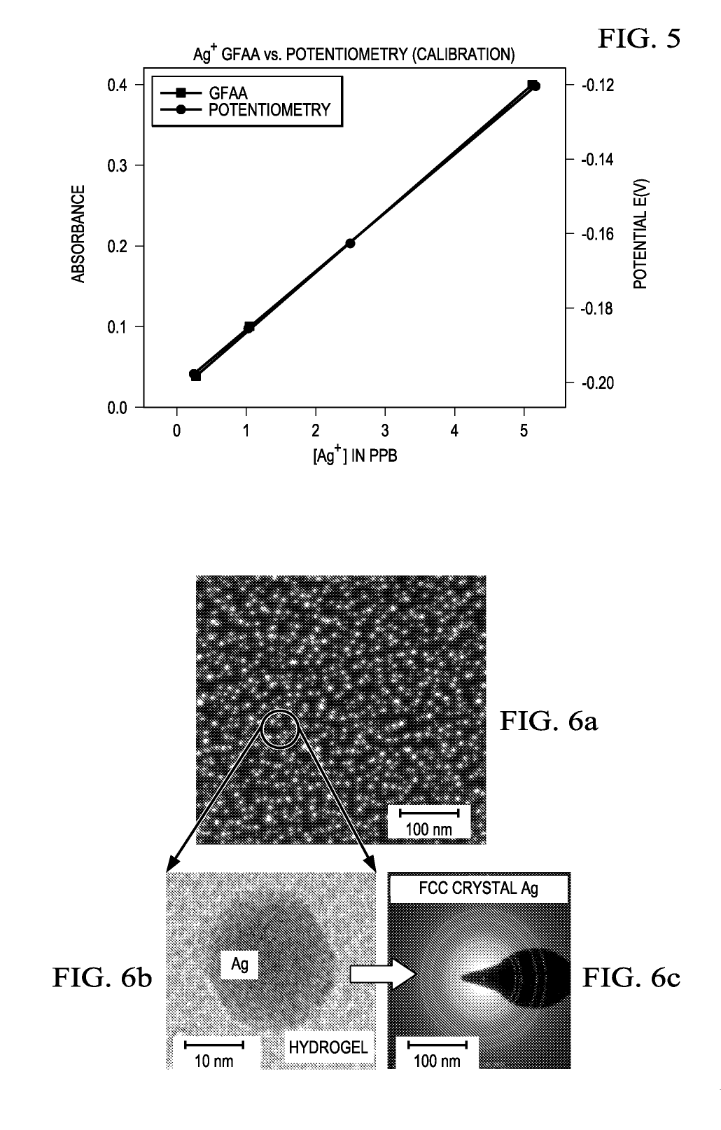

To further verify the absolute amounts of silver ions released from the composite films, graphite furnace atomic absorption (GFAA) spectroscopy was also carried out, using the standard silver ion solutions. In view of the high sensitivity of atomic absorption, the GFAA measurements were made with solutions that were dilutions of the potentiometry solutions, with concentrations ranging from 1 to 5 ppb as shown in FIG. 4. Again, an excellent linear correlation was observed between absorbance and Ag⁺ concentrations. Comparisons of GFAA results, using these diluted solutions, with the predicted Ag⁺ concentrations, calculated from the dilution process for the potentiometric samples provided confirmation of the quantitative agreement between the GFAA and emf data, as shown in FIG. 5. The excellent agreement between the two analytical techniques provided strong evidence that potentionmetric measurements accurately provide the absolute concentrations of the silver ions in solution.

An additional important analytical component of this work involved high resolution microscopic analyses of the silver-containing hydrogel films. In particular, these studies revealed the formation of uniformly sized small elemental silver nanoparticles, spontaneously formed in the films as a result of absorption of the silver ions into the hydrogels. An example of the silver nanoparticles, dispersed in the hydrogel film, is shown in the Transmission Electron Micrograph (TEM), FIG. 6 FIG. 6a, shows the well silver nanoparticles, as being extremely well dispersed and of relatively uniform size. Higher resolution TEM pictures of one of the nanoparticles, such as the one shown in FIG. 6b, reveals particle diameters of approximately 10 nm. Finally, X-Ray fluorescence emissions from these particles confirm the fact that they are indeed elemental silver nanoparticles in composition as shown in FIG. 6c. The spontaneous reduction of the Ag⁺ ions, from contact with the hydrogels, would be consistent with the presence of reducing agents, such as amine groups, present in the films. The presence of these silver nanoparticles, accompanied by their subsequent slow release and oxidation in solution back to silver ions play provide the long term, precisely controlled silver release, achieved by this invention, as documented below. The extent oxidation of the silver nanoparticles in an aqueous environment was measured with the potentiometric method described above. This study showed that the silver nanoparticles spontaneously oxidize in the presence of water and oxygen.

The silver release rates from a wide variety of composite hydrogel-silver loaded films were measured potentiometrically. These release rates were assessed as functions of film cross-link densities, film thickness, and with capping layers of differing compositions and thickness. Specific examples of these controlled release rates are shown below.

### Example 1

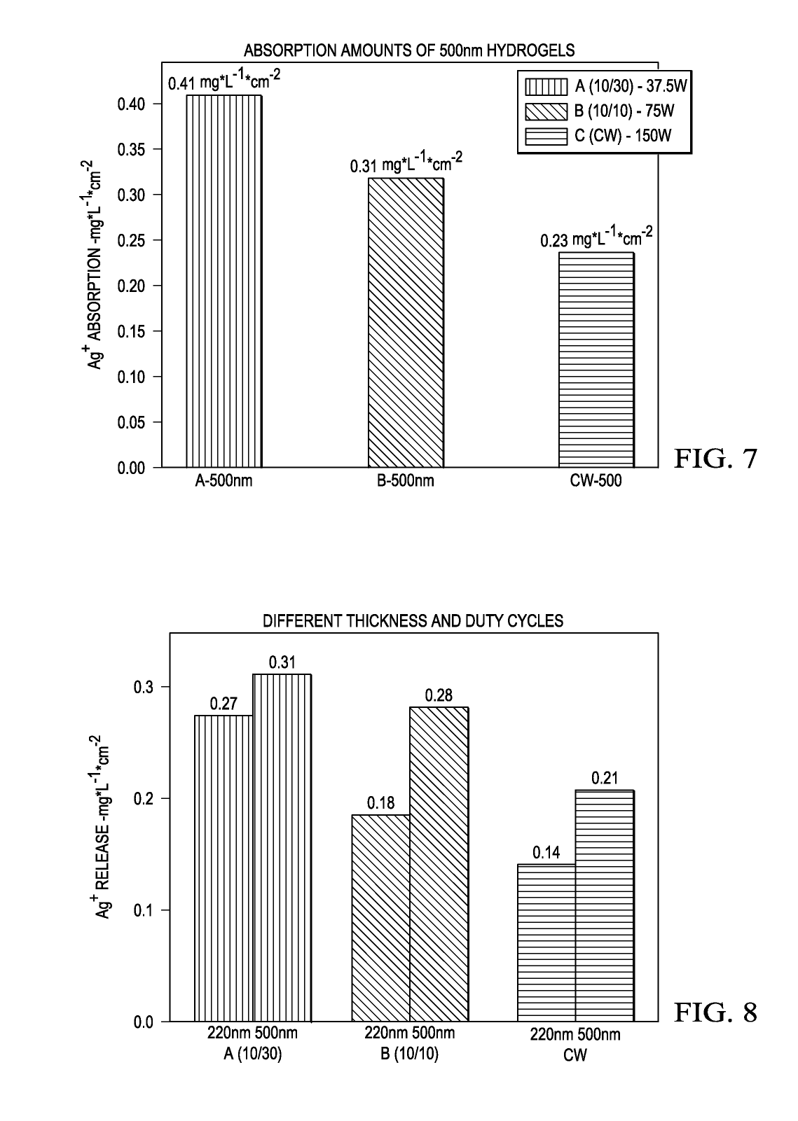

In this example, the inherent controllability of the cross-link density of the hydrogel films, synthesized by the plasma polymerization approach, is shown to provide excellent control of the initial extent of silver ion absorption by the films. For this purpose, several hydrogel films were synthesized by plasma polymerization of the monomer 1-amino-2-propanol (1A2P). Prior studies have clearly revealed that it is possible to vary the extent of film cross-link densities by simple changes in plasma parameters employed during the synthetic processes. In the present case, synthesis included film depositions under pulsed plasma conditions of 10 ms on and 30 ms off, 10 ms on and 10 ms off, and under continuous—wave (CW) operation. These samples are identified here, and in subsequent examples, as samples A, B and C, respectively. In all three cases, the peak power input was 150 Watts. In one set of experiments, the plasma deposition times were adjusted to produce 500 nm thick films for samples produced under each of these three deposition conditions. Following plasma synthesis, the films were subsequently immersed in Tris, pH 7.4 buffered solutions, each containing 10 mg of AgNO₃ per ml of solution. The solutions were maintained in contact with the films for a 24 hour period, to permit absorption of the silver nitrate. These films were subsequently analyzed to determine the extent of silver loading achieved. For this purpose, the Ag⁺ loaded films were first wet ashed (dissolved and digested in HNO₃/H₂O₂) and the resultant solution diluted to within range of 1-10 ppb and measured for [Ag+] by GFAA. From the GFAA adsorption data, and the known dilutions employed, the absolute concentrations of silver absorption by each film were calculated. FIG. 7 shows the variation of total Ag⁺ loading obtained for these 500 nm thick films of compositions A, B, and C. As shown in this figure, the extent of silver loading is dependent on the extent of polymer cross-linking in the films, decreasing from 0.41 to 0.31 to 0.23 mg·L⁻¹·cm⁻² as the cross-link density is increased in the order A, B and C, respectively. Thus, it is clear that the extent of initial silver ion absorption by the films is controllable by variations of the extent of film cross-link densities.

### Example 2

In this example, the thickness of the hydrogel films, which can be precisely controlled during the plasma synthesis step, provide an additional convenient way to control the extent of silver loading, and thus the effective drug or agent dosage available for delivery. In these experiments, films were synthesized for varying times under a fixed set of plasma operating conditions. It has been previously amply demonstrated that film thickness, particularly those deposited under pulsed plasma conditions, are a linear function of the plasma deposition times. After synthesis, the films were immersed in silver nitrate solution as described in Example 1. Subsequently, the accumulated silver release from films of differing thickness, observed over identical extended release periods, were measured potentiometrically. During these extended release periods, the solutions were periodically replaced and the hydrogel films immersed in buffered solutions devoid of silver. In this way any potential complications from re-absorption of the silver ions by the hydrogel films was avoided. It was observed that these accumulated releases, up to the end of the observation period, were indeed dependent on the film thickness employed. An example of this fact is shown in FIG. 8, for films of compositions A, B, and C, produced under the same three plasma conditions mentioned in example 1, having thicknesses of 220 and 500 nm. Clearly, the total accumulated silver release is dependent on film thickness. Additionally, this example further confirms the controllability of the silver loading and release as a function of film cross-link density as first noted in example 1.

### Example 3

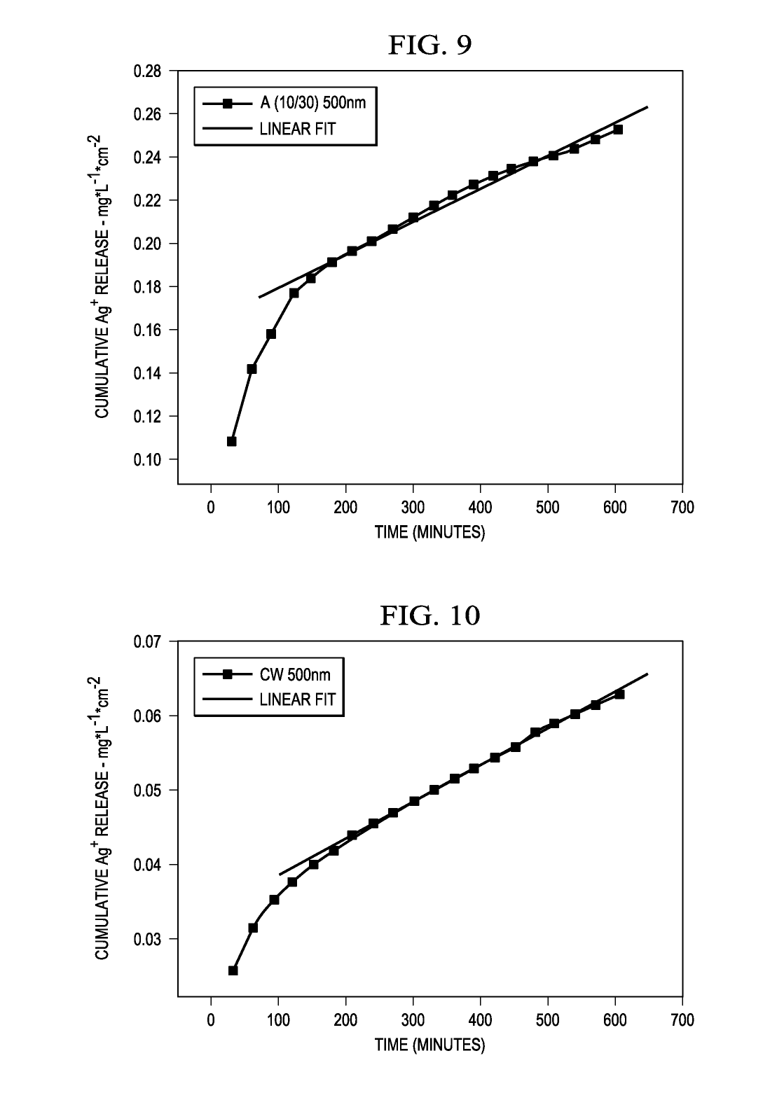

Studies of the kinetics of the initial release rates of the silver ions from these hydrogels were made as functions of both film compositions and film thickness. In all cases, it was observed that there is an initial significant release of silver, a “burst effect”, which is then followed by a steady release over an extended period of time. For each sample tested, it was observed that the extended release rates exhibited zero order kinetics. A typical example of the kinetics of the silver release is shown in FIG. 9 for measurements made with a composition A sample of 500 nm thickness. The initial burst effect is noted and the release rate during the initial 100 minutes is clearly changing with time. However, after this initial burst period, the release rates become essentially constant with time, thus exhibiting zero order kinetics.

### Example 4

The same general release profiles, as that described in Example 3, were observed with films of other thickness and other cross-link densities. For example, the release rates from 500 nm thick sample C films are shown in FIG. 10. Again an initial burst effect, followed by a steady release, was observed. The steady slow silver release, which again occurs after approximately 100 minutes, also exhibits zero order kinetics. It is significant to note that the both the magnitude of the initial burst release, and the subsequent release rates, are significantly lower for the C sample than that shown for the A sample in FIG. 9. In fact, both these effects are consistent with the prior examples and discussion in that sample C, deposited under CW plasma conditions, is significantly more cross-linked than sample A, which was synthesized under pulsed plasma conditions of 10 ms on and 30 ms off. As described above, the more cross-linked films absorb less silver ions when immersed in the silver nitrate solution and the release occurs more slowly as the cross-link density of the hydrogel film is increased. Examples 3 and 4 both provide added confirmation of controllability of agent release rates made available via the inherent controllability of the films cross-link densities and film thickness as made available by the plasma polymerization approach.

### Example 5

A further demonstration of the variation of silver release rates with film compositions is the measurement of the accumulated silver release from a set of 220 nm thick, sample A, B and C films. The total accumulated release was monitored over a period of approximately 3 days. As noted earlier, the films were periodically rinsed and then immersed in fresh, silver-free buffer solution, to minimize any re-absorption of initially released silver ions and to simulate the flow in biological systems.

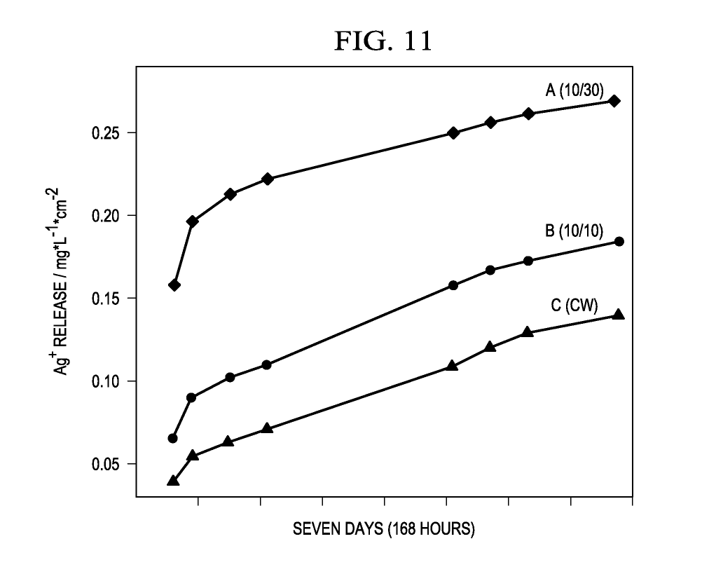

The results obtained are shown in FIG. 11. As before, the initial burst effect, followed by a slower, but constant, silver release is observed. With respect to FIG. 11, it is important to note that the abscissa is a non-linear time scale, used to more clearly emphasize key aspects of these graphs. As in the previous examples, the controllability of the overall agent release rates is clearly documented for this set of 220 nm films all produced from the same 1A2P monomer, but having different chemical compositions, as a result of changes in the plasma duty cycles employed during their synthesis.

### Example 6

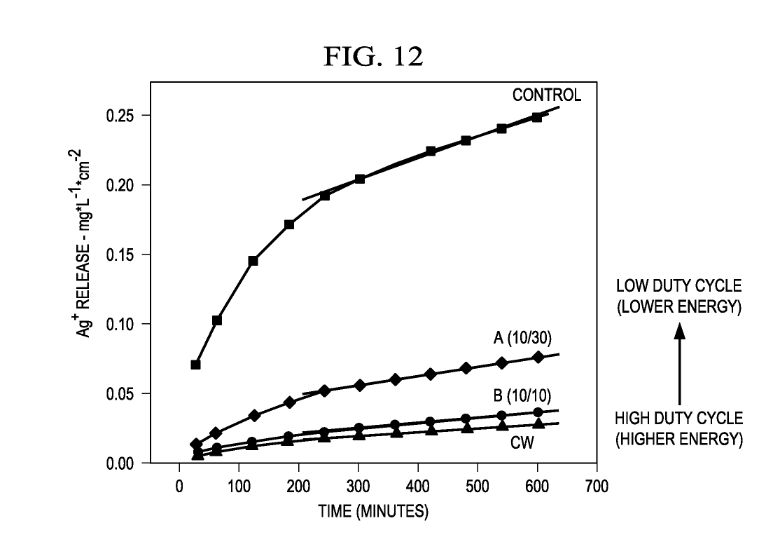

The deposition of a second film layer on top of the Ag⁺ loaded films, which henceforth is identify as a “capping” layer, was investigated as a possible additional controlled route to regulating the silver release rates from these composite films. In fact, the results obtained document the utility of this approach in not only controlling the Ag⁺ release rates but, additionally, the effectiveness of this second (capping) layer on modifying the initial release surge of silver, if so desired, when the samples are first immersed in the buffer solution. For example, in one set of experiments, four 500 nm samples of the composition A films were initially prepared and identically loaded with the silver ions. Keeping one of these samples as the control, the remaining three samples were then coated with a second capping layer of plasma polymerized 12 PA monomer having compositions A, B and C, respectively, each capping layer having a thickness of 500 nm. As shown in FIG. 12, it was observed that these capping layers were extremely effective in both reducing the extent of the initial burst of silver release, as well as reduction of the subsequent slower, zero order release rates, as compared to that of the uncapped control. Also, as documented in FIG. 12, and in accord with previously mentioned aspects of this invention involving correlation of release rates and film cross-link densities, the decrease in silver release from the capping layers becomes increasingly more pronounced in the sequence A<B<C, i.e. as the cross-link density increases.

### Example 7

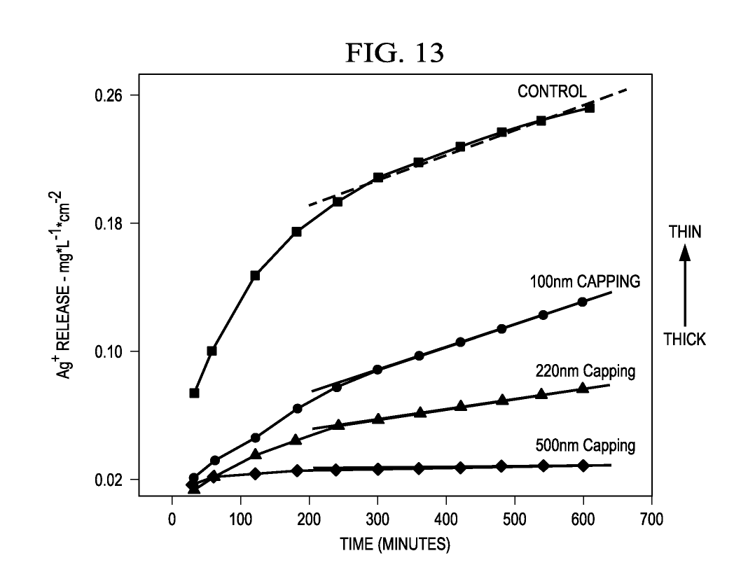

A further confirmation of the effectiveness of the capping layer approach was observed in which 500 nm thick silver loaded 1A2P films, of composition A, were capped with second layers of composition A having thicknesses of 100, 220 or t00 nm, respectively. Again, FIG. 13 demonstrates that both the initial surge release and subsequent slow zero order release of silver ions was decreased, relative to the uncapped 500 nm control. Furthermore, the magnitude of both effects were observed to be increasingly more pronounced as the thickness of the second capping layer was increased from 100 to 220 to 500 nm.

### Example 8

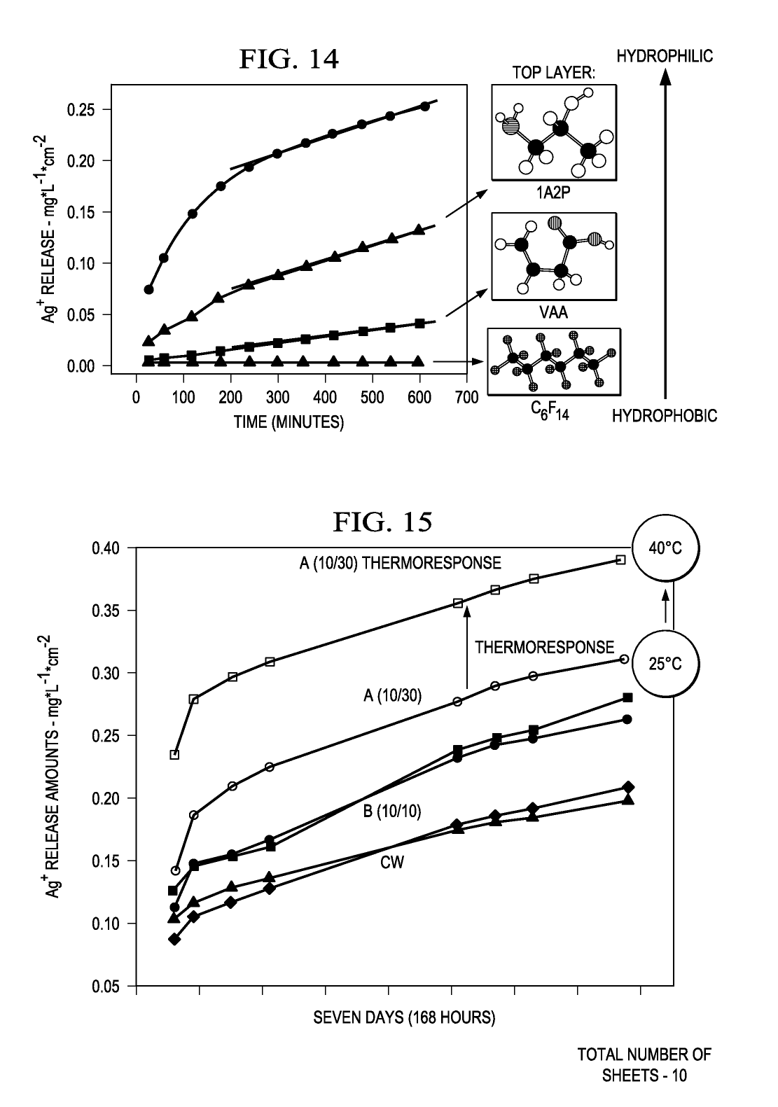

A further important example of the utility of the capping layer approach to provide ultra fine control of silver release rates was demonstrated by the use plasma polymerization of monomers other than 1A2P as capping layers. For these studies, polymer films from vinyl acetic acid (VAA) and perfluorohexane (PFH) monomers were plasma deposited on top of 500 nm thick 1A2P films of composition A. Each capping layer was of 500 nm thickness. The selection of polymer VAA and PFH as capping layers was based on the fact that these two films differ significantly in terms of surface energies, with the VAA film being the more wettable. The cumulative Ag⁺ release rates of these films were then measured over a time period of 600 minutes, with the results obtained shown in FIG. 13. For reference purposes, FIG. 14 also includes silver release rates from uncapped 500 nm composition A sample as well as from a 500 nm composition A film capped with a second layer of 500 nm composition A film. Several interesting observations can clearly be discerned from these results. First, the release rates of the Ag⁺ ions are substantially reduced after application of the capping layer, with the magnitude of this effect increasing in the order 1A2P<VAA<PFH. In fact, it is interesting to note that the PFH film essentially eliminates silver release over the entire time period employed. Also of interest, is the fact that the VAA film eliminates the initial surge release of silver, resulting in essentially zero order kinetics being observed over the entire observation period. Furthermore, based on the results of the prior examples, it is obvious that variation in the thickness of these second capping layers will provide additional silver release control, including, for example, much thinner PHF layers.

### Example 9

As previously reported, the 1A2P films, as well as hydrogels plasma synthesized from other low molecular weight monomers, exhibit thermal responsive behavior that results in a hydrogel transition from hydrophilic to more hydrophobic behavior with increasing temperature. The exact temperature at which these transitions are observed, known as the lower critical solution temperature (LCSTs), depends on the composition of the particular hydrogel. In fact, as previously reported, it is possible to synthesize plasma polymer films having tunable LCSTs by appropriate selection of monomer compositions and plasma deposition conditions.

As an integral component of the present invention, the effect of thermally induced hydrogel phase transitions on the silver release rates were examined. For this purpose, films of compositions A, B, and C, all of the same thickness, were prepared and soaked in silver nitrate solution. Following the 24 hour immersion, the films were rinsed and then examined with respect to silver release rates at temperatures of 25° and 40° C. These temperatures were chosen since it was known from prior work that the composition A hydrogel has a LCST of approximately 33° C., whereas films of compositions B and C do not exhibit LCSTs. The silver release rates observed for these various films at the two temperatures employed, as observed over a 7 day period, are shown in FIG. 14. As these results demonstrate, the silver release rate from the composition A film is significantly increased at the higher temperature whereas little change in release rates with temperatures was observed for the composition B and C films. These observations are consistent with the fact that only composition A films undergo the thermoresponsive structural transition over the temperature change employed. Furthermore, the results clearly indicate that this thermal responsive effect could be employed in regulating agent release rates with temperature variations.

Silver, silver ions, and silver compounds have been used for a somewhat more varied range of applications. Medical devices impregnated with both an antibiotic and a silver compound were discussed above. In addition, urinary catheters with a silver alloy/hydrogel coating have also been examined. Various vapor deposition methods have been employed to coat fabric and polymer/metal surfaces. No matter how the silver component is incorporated it can only work as a leaching agent because it only kills the cells after being taken up by the bacterium. Hence, any system utilizing silver will have diminishing effectiveness over time. The length of effectiveness can be increased by incorporating more silver, but at some point this becomes untenable. In addition, patient sensitivity to silver compounds and coatings has been reported.

Various hydantoin, also known as halamine, compounds have been successfully incorporated as polymer pendant groups or grafted to fabrics to impart antimicrobial action. Sun et al. have created a variety of hydantoin moieties and both incorporated them into polymer beads for water purification applications and grafted them onto various textiles to provide enhanced protection against bacteria. Worley et al. also created polymer beads with hydantoin pendant groups for water purification for comparison to polymer beads with quaternary ammonium pendant groups and found the hydantoin beads to be more effective. The hydantoin moieties are essentially storage compounds for chlorine, which is released to the impinging bacterium to kill it. Therefore, while not technically a leaching material, eventually the material is exhausted of antimicrobial protection and must be “recharged.” Often, this can be done by rinsing the fabric in a sodium hypochlorite solution. However, this makes the material undesirable for cases where long term protection is desired and recharging is not realistic. In addition, the amine-halogen bond is photosensitive, somewhat limiting the use of these materials.

“Contacting” as used herein refers to any means for providing the compounds of the invention to a surface to be protected from biofouling. Contacting can include spraying, wetting, immersing, dipping, painting, bonding or adhering or otherwise providing a surface with a compound of the invention.

Biofilm formation with health implications can involve those surfaces in all health-related environments, including surfaces found in medical environments and those surfaces in industrial or residential environments that are involved in those functions essential to well-being like nutrition, sanitation and the prevention of disease.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C1-C30 for straight chain, C3-C30 for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “carbocycle” is art-recognized and refers to an aromatic or non-aromatic ring in which each atom of the ring is carbon.

The term “nitro” is art-recognized and refers to —NO2; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO2.sup.-. “Halide” designates the corresponding anion of the halogens, and “pseudohalide”.

The terms “amine” and “amino” are art-recognized and refer to both unsubstituted and substituted amines, e.g., a moiety that may be represented by the general formulas:

The term “carbamoyl” refers to —O(C.dbd.O)NRR′, where R and R′ are independently H, aliphatic groups, aryl groups or heteroaryl groups.

The term “oxo” refers to a carbonyl oxygen.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen.

The composition of the polymer of the invention can vary. In certain embodiments, the polymers of the instant invention are hydrocarbon polymers, with significant hydrophobic character, and they contain at least one amino group with a pKa of greater than or equal to about 8. This means that, at conditions below a pH of 8, a significant portion of the amino groups will be protonated and cationic. Furthermore, in certain embodiments, the degree of polymer crosslinking can be controlled by adding a difunctional monomer or by increasing the energy input to the process. Crosslinking can increase the durability and adhesion of the coating without effecting the effectiveness. Cross-linking agents include, but are not limited to, 2-ethyl-2(hydroxymethyl)propane-trimethyacrylate (TRIM), acrylic acid, methacrylic acid, trifluoro-methacrylic acid, 2-vinylpyridine, 4-vinylpyridine, 3(5)-vinylpyridine, p-methylbenzoic acid, itaconic acid, 1-vinylimidazole, and mixtures thereof. Another aspect of the present invention relates to a composition, comprising a surface and a polymer coating, wherein said polymer coating comprises a plurality of monomers selected from the group consisting of styrenes and acrylates.

Another aspect of the present invention relates to a composition, comprising a surface and a polymer coating, wherein said polymer coating comprises a plurality of monomers selected from the group consisting of (dimethylaminomethyl)styrene, (dimethylaminoethyl)styrene, (diethylaminomethyl)styrene, (diethylaminoethyl)styrene, (dimethylaminomethyl)-.alpha.-methylstyrene, (diethylaminoethyl)acrylate, (dimethylaminoethyl)acrylate, (diethylaminomethyl)acrylate, (dimethylaminomethyl)acrylate, (dimethylaminopropyl)acrylate, (diethylaminoethyl)methacrylate, (dimethylaminoethyl)methacrylate, (diethylaminomethyl)methacrylate, (dimethylaminomethyl)methacrylate and (dimethylaminopropyl)methacrylate.

In addition to initiated chemical vapor deposition methodology, described below in detail, the antimicrobial polymer coatings of the invention may also be deposited using several other monomer and free radical initiating species: such as, plasma excitation without an initiator species (known generally as plasma-enhanced CVD) or photo-initiation of a UV sensitive initiator species (such as the peroxide or “azo” classes of molecules; e.g., t-butylperoxide or 2,2′-azobis(2-methylpropane)) or the monomer alone if the monomer is UV sensitive. Also, a method for enhancing coating bonding to the substrate, known generally as “grafting,” may be used to affix the antimicrobial polymers to a surface.

In one embodiment of the invention, an antimicrobial polymer coating is applied via initiated chemical vapor deposition (iCVD). Initiated chemical vapor deposition is capable of producing a range of polymeric and multifunctional nanocoatings. Coatings can be made extremely thin (down to about 10 nm) on objects with dimensions in the nanometer range (e.g., carbon nanotubes). Importantly, the object to be coated remains at room temperature, which means that nanothin coatings can be prepared on materials ranging from plastics to metals. The process is also conformal, which means it provides uniform coverage on objects which have small, complex, three-dimensional geometries.

Another aspect of the present invention relates to a method of coating a surface with a polymer, comprising the step of depositing a polymer on a surface using chemical vapor deposition; wherein said polymer coating comprises a plurality of monomers selected from the group consisting of (dimethylaminomethyl)styrene, (dimethylaminoethyl)styrene, (diethylaminomethyl)styrene, (diethylaminoethyl)styrene, (dimethylaminomethyl)-.alpha.-methylstyrene, (diethylaminoethyl)acrylate, (dimethylaminoethyl)acrylate, (diethylaminomethyl)acrylate, (dimethylaminomethyl)acrylate, (dimethylaminopropyl)acrylate, (diethylaminoethyl)methacrylate, (dimethylaminoethyl)methacrylate, (diethylaminomethyl)methacrylate, (dimethylaminomethyl)methacrylate and (dimethylaminopropyl)methacrylate.

There is large and growing interest in making antimicrobial a wide variety of materials and surfaces. Textiles and other materials present in a hospital setting have been shown to be sufficient bacterial supports, raising the possibility that these materials could be responsible for disease transfer among hospital populations.

A wide range of antimicrobial agents have been applied to surfaces: antibiotics including chlorhexidine, rifampin and monocycline and others, silver/silver ions/silver compounds, hydantoin (also known as halamine) compounds, furanone compounds, and quaternary ammonium or phosphonium polymers. There have been a smaller number of non-permanently cationic antimicrobial polymeric materials prepared for use on surfaces, generally incorporating benzoic acid derivatives

The various agents are most often physically applied to the surface, physically impregnated into the bulk of the material, or physically incorporated into a coating that is then applied to the surface for “controlled release”. In all these approaches the antimicrobial agent leaches from the surface, leading to two key problems: a limited time of effectiveness; and environmental, health and safety concerns, such as the promotion of drug resistant microbes. Non-leaching antimicrobial surfaces have been created by covalently grafting an antimicrobial polymer to the surface, atom transfer radical polymerization of an antimicrobial polymer directly from an initiating surface, and covalent attachment of an agent to a polymer chain. In the later case, any attachment scheme must not obscure the active moiety of the molecule. Also, particular care must be taken to ensure that the agent is actually covalently bound and is not just physically incorporated and that it is not releasing from the surface, which leads to the same issues discussed above for leaching antimicrobial agents.

The preferred perfluoro compound is a perfluorocarbon such as the most preferred perfluorinated trifluoromethyl substituted perfluorohexene. To form a perfluorinated surface also having a reactive surface, a perfluorinated compound is mixed with a carbonaceous compound having a reactive functional group such as an akenyl or alkyl halide, isothiocyanate, cyanide, benzene, acetate, mercaptan, glycidyl ether, ether, chloroformate, methyl sulfide, phenyl sulfone, phosphonic dichloride, trimethylsilane, triethoxysilane, acid, acid halide, amine, alcohol, or phosphide. The target materials may include any substance capable of reacting with the reactive functional groups. Preferred target materials include amino acids, fluorinated amino acids, proteins, peptides, saccharides, hormones, hormone receptors, polynucleotides, oligonucleotides, carbohydrates, glycosaminoglycans (such as heparin, for example) polyethylene glycol and polyethylene oxide.

Derivatives of all these various target materials may be prepared and still retain reactivity with one or more of the active functional groups such that they may be attached to an activated surface. In one aspect the present invention involves producing a surface with reduced adherence for biological materials. Surfaces with coupled polyethylene glycol, polyethylene oxide or abundant —CF3 groups are among the most preferred substituents for producing a surface with increased moisture protection, hydrophobicity and general stability. The use of a highly —CF3 substituted fluorocarbon monomer can yield exceptionally hydrophobic (or stable) surfaces via plasma deposition. For example, utilizing low duty cycle RF plasma deposition it is possible to retain, to a very high degree, the —CF3 content of the starting monomer.

Polymers that are suitable for use as the present invention include polyesters, polycarbonates, co-polymers of styrene and mixtures thereof. Examples of preferred matrix polymers are acrylonitrile-butadiene-styrene terpolymer (ABS); ABS modified polyvinylchloride; ABS-polycarbonate blends; acrylic resins and co-polymers: poly(methacrylate), poly(ethylmethacrylate), poly(methylmethacrylate), methylmethacrylate or ethylmethacrylate copolymers with other unsaturated monomers; casein; cellulosic polymers: ethyl cellulose, cellulose acetate, cellulose acetatebutyrate; ethyl vinyl acetate polymers and copolymers; poly(ethylene glycol); poly(vinylpyrrolidone); acetylated mono-, di-glycerides and tri-glycerides; poly(phosphazene); chlorinated natural rubber; polybutadiene; polyurethane; vinylidene chloride polymers and copolymers; styrene-butadiene copolymers; styrene-acrylic copolymers; alkylvinylether polymers and copolymers; cellulose acetate phthalates; epoxies; ethylene copolymers: ethylene-vinyl acetate-methacrylic acid, ethylene-acrylic acid copolymers; methylpentene polymers; modified phenylene oxides; polyamides; melamine formaldehydes; phenolformaldehydes; phenolic resins; poly(orthoesters); poly(cyanoacrylates); polydioxanone; polycarbonates; polyesters; polystyrene; polystyrene copolymers: poly(styrene-co maleic anhydride); urea-formaldehyde; urethanes; vinyl resins: vinyl chloride-vinyl acetate copolymers, polyvinyl chloride and mixtures of two or more of these.

Polymers that are suitable for use as the present invention include polyesters, polycarbonates, co-polymers of styrene and mixtures thereof. Examples of preferred matrix polymers are acrylonitrile-butadiene-styrene terpolymer (ABS); ABS modified polyvinylchloride; ABS-polycarbonate blends; acrylic resins and co-polymers: poly(methacrylate), poly(ethylmethacrylate), poly(methylmethacrylate), methylmethacrylate or ethylmethacrylate copolymers with other unsaturated monomers; casein; cellulosic polymers: ethyl cellulose, cellulose acetate, cellulose acetatebutyrate; ethyl vinyl acetate polymers and copolymers; poly(ethylene glycol); poly(vinylpyrrolidone); acetylated mono-, di-glycerides and tri-glycerides; poly(phosphazene); chlorinated natural rubber; polybutadiene; polyurethane; vinylidene chloride polymers and copolymers; styrene-butadiene copolymers; styrene-acrylic copolymers; alkylvinylether polymers and copolymers; cellulose acetate phthalates; epoxies; ethylene copolymers: ethylene-vinyl acetate-methacrylic acid, ethylene-acrylic acid copolymers; methylpentene polymers; modified phenylene oxides; polyamides; melamine formaldehydes; phenolformaldehydes; phenolic resins; poly(orthoesters); poly(cyanoacrylates); polydioxanone; polycarbonates; polyesters; polystyrene; polystyrene copolymers: poly(styrene-co maleic anhydride); urea-formaldehyde; urethanes; vinyl resins: vinyl chloride-vinyl acetate copolymers, polyvinyl chloride and mixtures of two or more of these.

Active pharmaceuticals ingredients Active Pharmaceutical Ingredients (APIs) may include analgesic anti-inflammatory agents such as, acetaminophen, aspirin, salicylic acid, methyl salicylate, choline salicylate, glycol salicylate, l-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, itraconazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

In one aspect of the present invention, coating materials such as perfluorohexane (C₆F₁₄), methyl methacrylate (MMA), and methacrylic acid (MAA) are provided. Coatings or polymer films obtained by plasma polymerization of methacrylic acid and methyl methacrylate are hydrophilic. Coatings or polymer films obtained by plasma polymerization of perfluorohexane are hydrophobic. Chemical structures of (a) perfluorohexane, (b) methyl methacrylate, and (c) methacrylic acid are shown below.

Perfluorocarbon compounds, such as perfluorohexane, yield plasma polymerized fluorinated films that exhibit good adhesion to many organic and inorganic substrates, have low intermolecular forces, low friction coefficient, and are biocompatible. The present inventors have previously shown that a pulsed plasma polymerization process may be used with perfluorocarbon compounds to create polymers and polymers films. (See U.S. Pat. No. 5,876,753; U.S. Pat. No. 6,306,506; U.S. Pat. No. 6,214,423; all of which are herein incorporated by reference). Polymers of hexafluoro-propylene oxide (C₃F₆O), perfluoro-2-butyltetrahydrofuran (PF₂BTHF, C₈F₁₆O) and perfluoropropylene (C₃F₆) create excellent coatings or films that are capable of attaching to substrate surfaces. Siloxane compounds, such as Hexamethyldisiloxane (HMDSO), also yield plasma polymerized films that exhibit good adhesion to many organic and inorganic substrates, have low intermolecular forces, low friction coefficient, hydrophobic behavior, and are biocompatible.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, itraconazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Plasma Enhanced Chemical Vapor Depositions PECVD provides for a solventless, pin-hole free, single-step encapsulation process in which the encapsulating or coating material may be modified depending on the process, itself. For example, the process is able to control encapsulation, and hence, particle introduction into an environment, by adjusting the side groups, thickness, wettability, molecular weight, cross-linking density, surface area and/or composition of the coating material.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed:

1. A process to synthesize hydrogels with precisely controlled levels of chemical compositions comprising the steps of:
mixing one or more monomers in a plasma reactor;
polymerizing the one or more monomers into a polymer;
crosslinking the polymer to form a hydrogel;
immersing the hydrogel in a first solution; and
adsorbing one or more solute species from the solution, wherein the one or more solute species are released at controlled rates.

2. The process of claim 1, further comprising the step of immersing the hydrogel in a second solution and adsorbing one or more capping agents from the second solution, wherein the one or more capping agents modify the release rate of the one or more solute species.

3. The process of claim 1, further comprising the step of adjusting a variable duty cycle pulsed plasma, a continuous wave plasma, or both to polymerize the one or more monomers into the polymer to control a polymer cross-link density and a thickness.

4. The process of claim 1, wherein the one or more solute species comprise one or more bioactive agents, active agents, drugs or antimicrobial agents.

5. The process of claim 1, wherein the one or more solute species comprise one or more silver ions that are spontaneously reduced to one or more elemental silver particles after adsorption by the hydrogel.

6. The process of claim 5, wherein the one or more elemental silver particles are nanosized particles, having diameters of 1-75 nm.

7. The process of claim 1, further comprising the steps of varying the one or more monomers, a plasma deposition variable or both to control a behavior at a lower critical solution temperature.

8. The process of claim 7, wherein the lower critical solution temperature can be controllably varied over the temperature range of approximately 30° C. to 60° C.

9. The process of claim 1, wherein the one or more capping agents has a surface energy ranging from highly non-wettable (hydrophobic) to highly wettable (hydrophilic).

10. The process of claim 1, wherein the one or more capping agents modify the release rate of the one or more solute species to eliminates an initial surge release of the one or more solute species.

11. A hydrogel made by the process of claim 1.

12. A hydrogel with precisely controlled levels of active agents comprising:
a mixture of one or more monomers polymerized in to a polymer in a plasma reactor;
one or more crosslinks connecting the polymer to form a hydrogel; and
one or more solute species associated with the polymer, wherein the one or more solute species are released at controlled rates from the polymer.

13. The hydrogel of claim 12, further comprising one or more capping agents connected to the one or more solute species, the polymer, or both to modify the release rate of the one or more solute species.

14. The hydrogel of claim 12, wherein a polymer cross-link density and a thickness are controlled by a duty cycle pulsed plasma, a continuous wave plasma, or both to polymerize the one or more monomers into the polymer.

15. The hydrogel of claim 12, wherein the one or more solute species comprise one or more bioactive agents, active agents, drugs or antimicrobial agents.

16. The hydrogel of claim 12, wherein the one or more solute species comprise one or more silver ions, that are spontaneously reduced to one or more elemental silver particles after adsorption by the hydrogel.

17. The hydrogel of claim 16, wherein the one or more elemental silver particles are nanosized particles, having diameters of 1-75 nm.

18. The hydrogel of claim 1, wherein the lower critical solution temperature behavior is controlled the one or more monomers, a plasma deposition variable or both.

19. The hydrogel of claim 22, wherein the lower critical solution temperature is controlled over the temperature range of approximately 30° C. to 60° C.
